(19) United States
(12) Patent Application Publication
(10) Pub. No.: US 2007/0196312 A1
(43) Pub. Date: Aug. 23, 2007

(54) Methods and kits for topical administration of hyaluronic acid

(76) Inventor: Dennis F. Gross, New York, NY (US)

Correspondence Address: KENYON & KENYON LLP, 1500 K STREET N.W., SUITE 700, WASHINGTON, DC, 20005, US

(21) Appl. No.: 11/375,023

(22) Filed: Mar. 15, 2006

Related U.S. Application Data
(60) Provisional application No. 60/774,549, filed on Feb. 21, 2006.
(60) Provisional application No. 60/776,922, filed on Feb. 28, 2006.

(51) Int. Cl. A61K 8/73

(52) U.S. Cl. 424/70.13

(57) ABSTRACT

The present invention provides a method for treating the skin of a subject comprising (a) applying an HA liquid to an area of the skin, wherein the HA liquid comprises hyaluronic acid, hyaluronate, cosmetically acceptable salts of hyaluronic acid, intramolecular esters of hyaluronic acid and/or intermolecular esters of hyaluronic acid; (b) massaging a self-heating mask composition into the area of the skin for a duration sufficient to activate heat, wherein the self-heating mask composition comprises at least one silicoaluminate; (c) letting the self-heating mask composition set on the area of the skin to form a mask; (d) optionally massaging the mask into the area of the skin; (e) optionally removing the mask from the area of the skin; and thereafter (f) optionally applying another skin care product to the area of the skin. The present invention also provides a kit useful for skin treatment, wherein the kit comprises the HA liquid and the self-heating mask composition comprises at least one silicoaluminate.

This patent application claims the benefits of U.S. Provisional Application Ser. No. ______ (serial number not assigned yet), entitled “Methods and Kits for Topical Administration of Hyaluronic Acid,” by Dennis Gross filed on Feb. 21, 2006 under Attorney Docket No. 10853/7 and U.S. Provisional Application Ser. No. ______ (serial number not assigned yet), entitled “Methods and Kits for Topical Administration of Hyaluronic Acid,” by Dennis Gross filed on Feb. 28, 2006 under Attorney Docket No. 10853/8.

The present invention is related to improved methods and kits for administering hyaluronic acid to the skin of a subject involving self heat generation and massage.

### BACKGROUND INFORMATION

Hyaluronic acid is a linear polysaccharide having repetitive alternate D-glucuronic acid and N-acetyl-D-glucosamine units, wherein each D-glucuronic acid unit is bonded to the immediately preceding N-acetyl-D-glucosamine unit via a β(1-4) linkage and each D-glucuronic acid unit is bonded to the following N-acetyl-D-glucosamine unit via a β(1-3) linkage. The disaccharide consisting of the D-glucuronic acid unit and the N-acetyl-D-glucosamine unit linked by the β(1-3) linkage is the basic building block of hyaluronic acid. Hyaluronic acid can be considered as a type of glycosaminoglycans or mucopolysaccharides. Hyaluronic acid molecules can consist of 250 to 25,000 pairs of D-glucuronic acid and N-acetyl-D-glucosamine units (Voet et al, Biochemistry, 1995, p. 264, John Wiley & Sons, Inc.). According to U.S. Pat. No. 6,838,086, hyaluronic acid molecules can consist of 2,000 to 10,000 pairs of D-glucuronic acid and N-acetyl-D-glucosamine units. The average molecular weight of hyaluronic acid can range from about 50,000 to about 8×10⁶ Daltons depending on the source of the hyaluronic acid and its method of isolation (U.S. Pat. No. 4,303,676). Molecular weight of up to 13×10⁶ has been reported for hyaluronic acid (U.S. Pat. Nos. 4,303,676 and 5,409,904). At physiological pH, the free carboxyl groups in the D-glucuronic acid units are ionized converting the acid form of hyaluronic acid to hyaluronate, which is anionic and can bind cations such as K⁺, Na⁺ and Ca⁺⁺.

Hyaluronic acid is a major component of the intercellular matrix and of the ground substance of connective tissues. Hyaluronic acid is also an integral component of complex proteoglycans. U.S. Pat. No. 6,946,551. Hyaluronic acid can be found in vitreous humor, synovial fluids, umbilical cord tissues, skin, rooster combs and certain Streptococcus species. Among animal tissues, soft connective tissues have the highest concentrations of hyaluronic acid. U.S. Pat. No. 4,141,973 discloses a process for obtaining ultra-pure, high molecular weight hyaluronic acid from animal tissues containing hyaluronic acid by removing blood from the animal tissues, extracting hyaluronic acid from the tissues, deproteinizing the extract and treating the extract with chloroform at pH 6.0-7.0 to remove any inflammation causing substances. U.S. Pat. No. 6,946,551 discloses a method of isolating hyaluronic acid from eggshell membrane. Commercially available hyaluronic acid is isolated from animal tissues such as mammalian umbilical cords and rooster combs, or obtained from the fermentation of certain hemolytic Streptococcal bacteria. Hyaluronic acid and alkali metal or ammonium salt thereof are generally available as a gel-like material (U.S. Pat. No. 5,409,904).

Hyaluronic acid can hold more water than other polymers and is probably responsible for the high water content of some tissues. U.S. Pat. No. 6,946,551. The large molecular size and large numbers of hydroxy groups and anionic carboxyl groups probably account for the water holding property of hyaluronic acid. Hyaluronic acid is viscoelastic in that hyaluronic acid solutions are quite viscous at low shear rate and the solutions flow more freely as the shear rate increases (Voet et al, Biochemistry, 1995, p. 264). Hyaluronic acid is a good lubricant in the body (e.g., hyaluronic acid in synovial fluids providing lubrication for synovial membranes) and it can also provide elasticity to joints. Partly due to the large number of mutually repellant anionic carboxyl groups, hyaluronate tends to form a rigid and highly hydrated molecule (Voet et al, Biochemistry, 1995, p. 264). Hyaluronic acid can provide rigidity to vertebrate disks (U.S. Pat. No. 6,946,551).

Intra-articular injection of a preparation containing hyaluronic acid of a molecular weight exceeding 3×10⁶ Daltons was disclosed to be useful in treating steroid arthropathy and progressive cartilage degeneration caused by proteoglycan degradation (U.S. Pat. No. 4,801,619). U.S. Pat. No. 6,607,745 discloses a method of relieving joint pain and musculoskeletal discomfort by oral ingestion of a composition comprising hyaluronic acid and an acceptable ingestible carrier. The method is useful for treating osteoarthritis and fibromyalgia. U.S. Pat. No. 5,409,904 discloses that hyaluronic acid compositions administered into the synovial space associate with a joint or tendon are useful in enhancing normal joint and tendon function by lubricating the joint and tendon against excess stress during movement.

U.S. Pat. No. 6,703,377 discloses a bone growth-promoting composition comprising hyaluronic acid and a growth factor such that the composition has a viscosity and biodegradability sufficient to persist at the site of desired bone growth for a duration enough to promote bone growth. The bone growth-promoting composition is injected through a syringe or catheter to the site of desired bone growth such as a bone fracture.

U.S. Pat. No. 6,509,322 discloses a pharmaceutical composition comprising hyaluronic acid, a pharmaceutically acceptable salt of hyaluronic acid, an ester of hyaluronic acid with an alcohol, intermolecular ester of hyaluronic acid or intramolecular ester of hyaluronic acid. The composition is useful for accelerating tissue repair in the treatment of burns, sores, ulcerations and wounds. The composition can be in the form of an aerosol, liquid spray, foam or dry spray. The composition is applied topically by spraying.

Hyaluronic acid has been reported to be useful in surgery. U.S. Pat. No. 5,140,016 discloses that using a solution comprising a hydrophilic, high molecular weight polymer such as hyaluronic acid having a molecular weight of at least about 500,000 Daltons to coat tissue surfaces and surgical instruments involved in surgery can prevent adhesions during surgery. According to U.S. Pat. No. 5,190,759, a solution containing hyaluronic acid having a molecular weight of 500,000 to 6×10⁶ Daltons is useful in preventing tissue adhesions following surgical procedures. Hyaluronic acid compositions introduced into a surgical site either during or after surgery are useful in preventing post-operative adhesion of healing tissues (U.S. Pat. No. 5,409,904). In a method of shortening the length of time required to complete a surgical procedure, an hyaluronic acid solution is used to coat tissue exposed at a surgical site (U.S. Pat. No. 6,541,460).

There are reports that hyaluronic acid is useful for the skin. U.S. Pat. No. 4,303,676 discloses a water-based, highly viscoelactic composition comprising water, a mixture of a low molecular weight hyaluronate (10,000 to 200,000 Daltons) and a high molecular weight hyaluronate (1×10⁶ to 4.5×10⁶ Daltons) in a ratio of 0.3 to 2.1 and protein derived from the natural material from which the hyaluronate is obtained. The composition is useful as a base for cosmetic formulations and also has emollient, moisturizing, elasticizing and lubricating properties when applied to the skin (U.S. Pat. No. 4,303,676).

U.S. Pat. No. 5,571,503 discloses a cosmetic composition for protecting the skin from several damaging components of environmental pollution, providing protection against moisture loss, and protecting the skin against damage due to free radical activity and UV light. The cosmetic composition comprises a sunscreen, an anti-pollution complex, a micellar complex containing sodium hyaluronate and an anti-free radical complex containing melanin, vitamin E or vitamin C.

According to U.S. Pat. No. 5,728,391, hyaluronic acid having a low molecular weight fails to provide adequate moisturizing effect in skin care compositions. Hyaluronic acid having a molecular weight of at least 4×10⁶ tends to be highly viscous, making formulation difficult. U.S. Pat. No. 5,728,391 discloses an agent containing hyaluronic acid having an average molecular weight of 800,000 to 4×10⁶, useful for treating a skin disease selected from contact dermatitis, xerosis senilis, asteatosis, eczema, miliaria and diaper rash.

U.S. Pat. No. 6,806,259 discloses a soft gelatin formulation comprising low molecular weight hyaluronic acid, preferably having molecular weight between 50,000 to 200,000 Daltons. The formulation is administered orally for use as a nutritional supplement to provide the primary benefit of causing skin softening.

U.S. Pat. No. 6,689,349 discloses a composition comprising fragments of hyaluronic acid produced by means of enzymes from hyaluronic acid, pharmaceutical carriers and auxiliary substances. The composition is useful in protecting the skin and preventing traumatic symptoms, inflammation and aging due to environmental factors or illnesses. The composition is formulated in the form of a paste, ointment, cream, emulsion, gel, stick, colloidal carrier system or solution.

U.S. Pat. No. 6,890,901 discloses a pharmaceutical composition comprising a mixture of hyaluronic acid and liposomes encapsulating a pharmaceutically active substance effective to treat skin disorder, wherein the pharmaceutical composition is administered topically to deliver the pharmaceutically active substance in the dermis or sub-dermis while minimizing systemic circulation of the substance.

Even though the beneficial effects of hyaluronic acid to the skin have been known for some time and a number of dermatological products containing hyaluronic acid are commercially available, there is still a need for an improved method and kit for administering hyaluronic acid to the skin which is both highly effective and relatively easy to use. The present invention meets such a need with a novel method of administering a composition containing hyaluronic acid to the skin, and a kit suitable for use in the novel method.

### SUMMARY OF THE INVENTION

The present invention provides a method for administering hyaluronic acid and/or hyaluronate to the skin of a subject or for treating the skin of a subject comprising

(a) applying an HA liquid to an area of the skin, wherein the HA liquid comprises HA, and wherein HA is at least one substance selected from the group consisting of hyaluronic acid, hyaluronate, cosmetically acceptable salts of hyaluronic acid, intramolecular esters of hyaluronic acid and intermolecular esters of hyaluronic acid;

(b) applying a self-heating mask composition into the area of the skin to generate heat locally, wherein the self-heating mask composition comprises at least one silicoaluminate, preferably sodium silicoaluminate, and wherein the self-heating mask composition is preferably massaged into the area of the skin to promote heat generation;

(c) letting the self-heating mask composition set on the area of the skin to form a mask;

(d) optionally massaging the mask into the area of the skin;

(e) optionally removing the mask from the area of the skin; and thereafter

(f) optionally applying another skin care product to the area of the skin.

The present invention also provides a kit useful for skin treatment or administering hyaluronic acid and/or hyaluronate, wherein the kit comprises an HA liquid and a self-heating mask composition, and wherein the HA liquid comprises HA and the self-heating mask composition comprises sodium silicoaluminate. Preferably, the kit further comprises at least one device, e.g., brush or pad, for applying the HA liquid and/or the self-heating mask composition to the skin.

The method and kit of the present invention are useful in allowing consumers to achieve professional skin care results at home in a small number of easy steps. The HA liquid, which may contain a cocktail of antioxidants, can plump trouble areas minimizing fine lines and wrinkles caused by dehydration. The self-heating mask composition can create a barrier on the skin increasing circulation and forcing optional antioxidant and collagen stimulating ingredients deep into the skin to achieve more beneficial effects from the antioxidant and collagen simulating ingredients. Skin treatment using the method and/or kit of the invention can leave dry, dehydrated skin immediately plumper, smoother and younger-looking.

The optional skin care product applied in step (f) can be, but is not limited to, a moisturizer, a skin peel product such as an acid/alkaline skin peel product disclosed in U.S. patent application Ser. No. 09/338,729 filed on Jun. 23, 1999, the disclosure of which is herein incorporated by reference, Antioxidant Firming Face Serum and/or Anti-Aging Vitamin C Gel. Alternatively, the moisturizer, skin peel product, Antioxidant Firming Face Serum and/or Anti-Aging Vitamin C Gel can be applied to the skin before the application of the method or kit of the present invention.

Before and/or after the application of the method or kit of the present invention to the skin, the skin can optionally be subjected to one or more skin care treatments, e.g., facial, acne treatment, acne prevention, and skin peel, performed by a professional or consumer. For instance, the one or more skin care treatments can be performed to the skin within 12 hours, preferably within 6 hours, more preferably within 3 hours, further more preferably within 1 hour, even more preferably within 30 minutes, and most preferably within 15 minutes, before and/or after the application of the method or kit of the present invention to the skin.

### DETAILED DESCRIPTION OF THE INVENTION

In the description of the present invention, the term “HA” means, unless otherwise indicated, at least one substance selected from the group consisting of hyaluronic acid, hyaluronate, cosmetically acceptable salts of hyaluronic acid, intramolecular esters of hyaluronic acid and intermolecular esters of hyaluronic acid.

The term “intramolecular esters of hyaluronic acid” refers to esters formed by the reaction of at least one carboxyl group in a molecule of hyaluronic acid or hyaluronate with at least one hydroxy group in the same molecule of hyaluronic acid or hyaluronate.

The term “intermolecular esters of hyaluronic acid” refers to esters formed by the reaction of at least one carboxyl group in a molecule of hyaluronic acid or hyaluronate with at least one hydroxy group in another molecule of hyaluronic acid or hyaluronate.

The term “subject” means a mammal, preferably a human. The subject can be a human consumer or patient. More preferably, the subject is a human consumer.

As used herein, the term “cosmetically acceptable” modifying a substance means that the substance is of sufficiently high purity and suitable for use in contact with human skin without undue toxicity, incompatibility and instability. A “cosmetically acceptable” substance, preferably, causes little or no allergic response.

The term “cosmetically acceptable salts of hyaluronic acid” includes sodium, potassium, lithium, calcium, magnesium, aluminum, zinc and ammonium salts of hyaluronic acid. Preferably, the term “cosmetically acceptable salts of hyaluronic acid” means sodium salt of hyaluronic acid.

The HA liquid comprises HA. Preferably, the HA liquid further comprises at least one cosmetically acceptable vehicle or carrier. The HA liquid may also include at least one emollient/humectant/moisturizer and/or at least one cosmetically acceptable excipient. The HA liquid may also include at least one additional active ingredient. The HA liquid may also include at least one aesthetic component.

The self-heating mask composition comprises at least one silicoaluminate. Examples of “silicoaluminate” include sodium silicoaluminate, potassium silicoaluminate, calcium silicoaluminate, magnesium silicoaluminate and zinc silicoaluminate.

The self-heating mask composition, preferably, further comprises at least one emollient/humectant/moisturizer (e.g., butylene glycol). The self-heating mask composition may further comprise at least one additional active ingredient, at least one aesthetic component, at least one cosmetically acceptable excipient and/or at least one cosmetically acceptable vehicle or carrier.

The methods or kits of the present invention are also useful in potentiating or enhancing the effects of the at least one emollient/humectant/moisturizer, at least one cosmetically acceptable excipient and/or at least one additional active ingredient present in the HA composition and/or the self-heating mask composition.

The at least one cosmetically acceptable vehicle or carrier is, preferably, water or a cosmetically acceptable aqueous buffer having a pH of about 7.0 to about 7.4, and, more preferably, the water or aqueous buffer is purified and/or sterile.

The at least one cosmetically acceptable excipient is selected from the group consisting of surfactant/emulsifying agents, absorbents, antifoaming agents, binders, biological additives, chelating agents, denaturants, preservatives, solubilizing agents, solvents and thickening agents.

The at least one additional active ingredient is selected from the group consisting of antioxidants, free-radical scavengers, antimicrobial agents, topical analgesics, steroidal anti-inflammatory drugs, anti-acne agents, reducing agents, vitamins, skin protecting agents, skin bleaching agents, skin conditioning agents, skin soothing agents, skin healing agents, green tea extract, P. emblica (Amla), arnica, chamomile extract and cucumber extract. The at least one additional active ingredient is, preferably, at least one antioxidant.

Suitable surfactant/emulsifying agents include ceteareths, ceteths, laneths, laureths, isoseareths, steareths, cetyl alcohol, deceths, dodoxynols, glyceryl palmitate, glyceryl stearate, laneths, myreths, nonoxynols, octoxynols, oleths, PEG-castor oil, poloxamers (e.g., poloxamer 407), poloxamines, polysorbates, sodium laurate, ammonium laureth sulfate, sodium laureth sulfate, sodium lauroyl sarcosinate, sodium lauroyl taurate, sodium lauryl sulfate, sodium methyl cocoyl taurate, sodium methyl oleoyl taurate, sodium nonoxynol sulfate, sodium cetyl sulfate, sodium cetearyl sulfate, sodium cocoate, sodium cocoyl isethionate and sodium cocoyl sarcosinate. Other suitable surfactant/emulsifying agents would be known to one of skill in the art and are listed in the CTFA International Cosmetic Ingredient Dictionary and Handbook, Vol. 2, 7 th Edition (1997). Preferred surfactants include octoxynol-9 and polysorbate-20.

Examples of chelating agents are disodium EDTA, trisodium EDTA, tetrasodium EDTA and sodium metasilicate.

Examples of suitable preservatives include imidazolidinyl urea, diazolidinyl urea, phenoxyethanol, methylparaben, ethylparaben and propylparaben.

Examples of thickening agents include isopropyl myristate, isopropyl palmitate, isodecyl neopentanoate, squalene, mineral oil, C₁₂-C₁₅ benzoate and hydrogenated polyisobutene.

Examples of antioxidants and free-radical scavengers include ascorbic acid, salts of ascorbic acid such as ascorbyl palmitate and sodium ascorbate, ascorbyl glucosamine, vitamin E (i.e., tocopherols such as a-tocopherol), derivatives of vitamin E (e.g., tocopheryl acetate), retinoids such as retinoic acid, retinol, trans-retinol, cis-retinol, mixtures of trans-retinol and cis-retinol, 3-dehydroretinol and derivatives of vitamin A (e.g., retinyl acetate, retinal and retinyl palmitate, also known as tetinyl palmitate), sodium citrate, sodium sulfite, lycopene, anthocyanids, bioflavinoids (e.g., hesperitin, naringen, rutin and quercetin), superoxide dismutase, glutathione peroxidase, butylated hydroxytoluene (BHT), butylated hydroxyanisole (BHA), indole-3-carbinol, pycnogenol, melatonin, sulforaphane, pregnenolone, lipoic acid and 4-hydroxy-5-methyl-3[2H]-furanone.

The antimicrobial agents are antibacterial agents and antifungal agents. Examples of the antimicrobial agents include benzoyl peroxide, erythromycin, tetracycline, triclosan, azelaic acid, clindamycin, chlorhexidine, neomycin, miconazole and clotrimazole.

Examples of topical analgesics include aspirin and non-steroidal anti-inflammatory drugs.

Suitable non-steroidal anti-inflammatory drugs include ibuprofen, naproxen, benoxaprofen, flurbioprofen, fenoprofen, fenbufen, ketoprofen, indoprofen, pirprofen, carprofen, oxaprozin, pranoprofen, microprofen, tioxaprofen, suprofen, alminoprofen, tiaprofenic acid, fluprofen and bucloxic acid.

The skin conditioning agents can be emollients, humectants and moisturizers, which include urea; guanidine; aloe vera; glycolic acid and glycolate salts such as ammonium and quaternary alkyl ammonium; lactic acid and lactate salts such as sodium lactate, ammonium lactate and quaternary alkyl ammonium lactate; polyhydroxy alcohols such as sorbitol, glycerol, hexanetriol, propylene glycol, butylene glycol, hexylene glycol, polyethylene glycol; carbohydrates such as alkoxylated glucose; starches; starch derivatives; glycerin; pyrrolidone carboxylic acid (PCA); lactamide monoethanolamine; acetamide monoethanolamine; volatile silicone oils; nonvolatile silicone oils; and mixtures thereof. Suitable silicone oils can be polydialkylsiloxanes, polydiarylsiloxanes, polyalkarylsiloxanes and cyclomethicones having 3 to 9 silicon atoms.

Skin soothing agents include bisabolol.

Suitable steroidal anti-inflammatory drugs include, for example, hydrocortisone and bensonide.

Suitable anti-acne agents can by drying agents, keratolyic agents, epidermolytic agents, antimicrobial agents and retinoids. Examples of anti-acne agents include sulfur, resorcinol, glycolic acid, lactic acid, pyruvic acid, salicylic acid, retinoic acid, derivatives of retinoic acid, and tetracycline.

The skin protecting agents are agents that protect the skin against chemical irritants and/or physical irritants, e.g., UV light, including sunscreens, anti-acne additives, anti-wrinkle and anti-skin atrophy agents. Suitable sunscreens as skin protecting agents include 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropy dibenzoylmethane, 3-benzylidene camphor, 3-(4-methylbenzylidene) camphor, anthanilates, ultrafine titanium dioxide, zinc oxide, iron oxide, silica, 4-N,N-(2-ethylhexyl)methylaminobenzoic acid ester of 2,4-dihydroxybenzophenone, 4-N,N-(2-ethylhexyl)-methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone and 4-N,N(2-ethylhexyl)-methylaminobenzoic acid ester of 4-(2-hydroxyethoxy)dibenzoylmethane. Suitable anti-acne agents include salicylic acid; 5-octanoyl salicylic acid; resorcinol; retinoids such as retinoic acid and its derivatives; sulfur-containing D and L amino acids other than cysteine; lipoic acid; antibiotics and antimicrobials such as benzoyl peroxide, octopirox, tetracycline, 2,4,4′-trichloro-2′-hydroxydiphenyl ether, 3,4,4′-trichlorobanilide, azelaic acid, phenoxyethanol, phenoxypropanol, phenoxisopropanol, ethyl acetate, clindamycin and melclocycline; flavonoids; and bile salts such as scymnol sulfate, deoxycholate and cholate. Examples of anti-wrinkle and anti-skin atrophy agents are retinoic acid and its derivatives, retinol, retinyl esters, salicylic acid and its derivatives, sulfur-containing D and L amino acids except cysteine, alpha-hydroxy acids (e.g., glycolic acid and lactic acid), phytic acid, lipoic acid and lysophosphatidic acid.

Suitable skin bleaching agents include, for example, hydroquinone, kojic acid and sodium metabisulfite.

The at least one aesthetic agent can be at least one of fragrances, pigments, colorants, essential oils, skin sensates and astringents. Suitable aesthetic agents include clove oil, menthol, camphor, eucalyptus oil, eugenol, methyl lactate, bisabolol, witch hazel distillate (preferred) and green tea extract (preferred).

In some of the embodiments of the present invention, the HA liquid comprises HA, at least one anti-oxidant, at least one skin conditioning agent, at least one reducing agent, at least one additional active agent, at least one cosmetically acceptable vehicle or carrier and at least one cosmetically acceptable excipient. The HA can be hyaluronic acid and/or hyaluronate, preferably sodium hyaluronate. The at least one anti-oxidant can be ascorbic acid, ascorbyl palmitate, ascorbyl glucosamine, tocopheryl acetate, retinyl palmitate, superoxide dismutase, or mixtures thereof. The at least one skin conditioning agent can be cyclomethicone and/or dimethiconol. The at least one reducing agent can be ubiquinone. The at least one additional active agent can be Camellia sinensis leaf extract and/or white tea extract or juice, preferably Camellia sinensis leaf extract. The at least one cosmetically acceptable vehicle or carrier can be purified or sterile water, preferably purified water. The at least one cosmetically acceptable excipient can be phospholipids.

In one of the embodiments of the present invention, the HA liquid comprises cyclomethicone, dimethiconol, sodium hyaluronate, Camellia sinensis leaf extract, ascorbyl palmitate, ascorbyl glucosamine, tocopheryl acetate, retinyl palmitate, superoxide dismutase, ubiquinone, phospholipids and purified water with the optional inclusion of ascorbic acid.

In one of the embodiments of the present invention, the HA liquid comprises the following ingredients:

| Ingredient | Amount (Weight %) |
|---|---|
| cyclomethicone | about 0% to about 99%, |
| dimethiconol | about 0% to about 30%, |
| sodium hyaluronate | about 0.005% to about 99.5%, |
| Camellia sinensis leaf extract | about 0% to about 20%, |
| optional ascorbic acid | about 0% to about 20%, |
| ascorbyl palmitate | about 0% to about 20%, |
| ascorbyl glucosamine | about 0% to about 20%, |
| tocopheryl acetate | about 0% to about 20%, |
| retinyl palmitate | about 0% to about 20%, |
| superoxide dismutase | about 0% to about 1%, |
| ubiquinone | about 0% to about 20%, |
| phospholipids | about 0% to about 20% and |
| purified water | about 0% to about 50%. |

In the embodiment, the HA liquid preferably comprises the following ingredients:

| Ingredient | Amount (Weight %) |
|---|---|
| cyclomethicone | about 5% to about 95%, |
| dimethiconol | about 5% to about 25%, |
| sodium hyaluronate | about 0.01% to about 85%, |
| Camellia sinensis leaf extract | about 0.01% to about 10%, |
| optional ascorbic acid | about 0.01% to about 10% (if present), |
| ascorbyl palmitate | about 0.01% to about 10%, |
| ascorbyl glucosamine | about 0.01% to about 10%, |
| tocopheryl acetate | about 0.01% to about 10%, |
| retinyl palmitate | about 0.01% to about 10%, |
| superoxide dismutase | about 0.0001% to about 0.1%, |
| ubiquinone | about 0.01% to about 10%, |
| phospholipids | about 0.01% to about 10% and |
| purified water | about 0.01% to about 20%. |

In the embodiment, the HA liquid more preferably comprises the following ingredients:

| Ingredient | Amount (Weight %) |
|---|---|
| cyclomethicone | about 10% to about 90%, |
| dimethiconol | about 10% to about 20%, |
| sodium hyaluronate | about 0.01% to about 70%, |
| Camellia sinensis leaf extract | about 0.01% to about 5%, |
| optional ascorbic acid | about 0.01% to about 5% (if present), |
| ascorbyl palmitate | about 0.01% to about 5%, |
| ascorbyl glucosamine | about 0.01% to about 5%, |
| tocopheryl acetate | about 0.01% to about 5%, |
| retinyl palmitate | about 0.01% to about 5%, |
| superoxide dismutase | about 0.0001% to about 0.01%, |
| ubiquinone | about 0.01% to about 5%, |
| phospholipids | about 0.01% to about 5% and |
| purified water | about 0.01% to about 10%. |

In the embodiment, the HA liquid even more preferably comprises the following ingredients:

| Ingredient | Amount (Weight %) |
|---|---|
| cyclomethicone | about 86.90%, |
| dimethiconol | about 13.00%, |
| sodium hyaluronate | about 0.01%, |
| Camellia sinensis leaf extract | about 0.01%, |
| optional ascorbic acid | about 0.01% (if present), |
| ascorbyl palmitate | about 0.01%, |
| ascorbyl glucosamine | about 0.01%, |
| tocopheryl acetate | about 0.01%, |
| retinyl palmitate | about 0.01%, |
| superoxide dismutase | about 0.0001%, |
| ubiquinone | about 0.02%, |
| phospholipids | about 0.01% and |
| purified water | about 0.01%. |

In some of the embodiments of the present invention, the self-heating mask composition comprises at least one silicoaluminate, preferably sodium silicoaluminate, at least one skin conditioning agent, at least one surfactant, at least one additional active agent, at least one anti-oxidant, at least one cosmetically acceptable excipient and at least one aesthetic agent, and optionally at least one cosmetically acceptable vehicle or carrier. The at least one skin conditioning agent can be butylene glycol and/or petrolatum. The at least one surfactant can be PEG-8 and/or methyl GLUCETH-20. The at least one additional active agent can be Camellia sinensis leaf extract and/or white tea extract or juice. The at least one anti-oxidant can be ascorbyl palmitate, retinyl palmitate, tocopheryl acetate, or mixtures thereof. The at least one cosmetically acceptable excipient can be hydroxypropylcellulose and/or hydroxypropyl methylcellulose.

In one of the embodiments of the present invention, the self-heating mask composition comprises sodium silicoaluminate, butylene glycol, PEG-8, Camellia sinensis leaf extract, white tea extract or juice, ascorbyl palmitate, retinyl palmitate, tocopheryl acetate, dimethicone, methyl GLUCETH-20, hydroxypropylcellulose, hydroxypropyl methylcellulose, petrolatum and titanium dioxide, with optional inclusion of phospholipids.

In one of the embodiments of the present invention, the self-heating mask composition comprises the following ingredients:

| Ingredient | Amount (weight %) |
|---|---|
| sodium silicoaluminate | about 2% to about 99%, |
| butylene glycol | about 0% to about 98%, |
| PEG-8 | about 0% to about 25%, |
| Camellia sinensis leaf extract | about 0% to about 20%, |
| white tea extract or juice | about 0% to about 20%, |
| ascorbyl palmitate | about 0% to about 20%, |
| retinyl palmitate | about 0% to about 20%, |
| tocopheryl acetate | about 0% to about 20%, |
| dimethicone | about 0% to about 20%, |
| methyl GLUCETH-20 | about 0% to about 20%, |
| hydroxypropyl cellulose | about 0% to about 20%, |
| hydroxypropyl methylcellulose | about 0% to about 20%, |
| petrolatum | about 0% to about 20%, |
| titanium dioxide | about 0% to about 20% and |
| optional phospholipids | about 0% to about 20%. |

In the embodiment, the self-heating mask composition preferably comprises the following ingredients:

| Ingredient | Preferred Amount (Weight %) |
|---|---|
| sodium silicoaluminate | about 10% to about 80%, |
| butylene glycol | about 10% to about 80%, |
| PEG-8 | about 0.1% to about 10%, |
| Camellia sinensis leaf extract | about 0.01% to about 10%, |
| white tea extract or juice | about 0.01% to about 10%, |
| ascorbyl palmitate | about 0.01% to about 10%, |
| retinyl palmitate | about 0.01% to about 10%, |
| tocopheryl acetate | about 0.01% to about 10%, |
| dimethicone | about 0.01% to about 10%, |
| methyl GLUCETH-20 | about 0.01% to about 10%, |
| hydroxypropylcellulose | about 0.01% to about 10%, |
| hydroxypropyl methylcellulose | about 0.01% to about 10%, |
| petrolatum | about 0.1% to about 10%, |
| titanium dioxide | about 0.01% to about 10% and |
| optional phospholipids | about 0.01% to about 10% (if present). |

In the embodiment, the self-heating mask composition more preferably comprises the following ingredients:

| Ingredient | More Preferred Amount (Weight %) |
|---|---|
| sodium silicoaluminate | about 20% to about 70%, |
| butylene glycol | about 20% to about 70%, |
| PEG-8 | about 0.5% to about 5%, |
| Camellia sinensis leaf extract | about 0.01% to about 2%, |
| white tea extract or juice | about 0.01% to about 2%, |
| ascorbyl palmitate | about 0.01% to about 2%, |
| retinyl palmitate | about 0.01% to about 2%, |
| tocopheryl acetate | about 0.01% to about 2%, |
| dimethicone | about 0.01% to about 2%, |
| methyl GLUCETH-20 | about 0.01% to about 2%, |
| hydroxypropylcellulose | about 0.01% to about 2%, |
| hydroxypropyl methylcellulose | about 0.01% to about 2%, |
| petrolatum | about 0.5% to about 5%, |
| titanium dioxide | about 0.01% to about 2% and |
| optional phospholipids | about 0.01% to about 2% (if present). |

In the embodiment, the self-heating mask composition even more preferably comprises the following ingredients:

| Ingredient | Amount (Weight %) |
|---|---|
| sodium silicoaluminate | about 31.84%, |
| butylene glycol | about 62.02%, |
| PEG-8 | about 3.35%, |
| Camellia sinensis leaf extract | about 0.10%, |
| white tea extract or juice | about 0.01%, |
| ascorbyl palmitate | about 0.01%, |
| retinyl palmitate | about 0.01%, |
| tocopheryl acetate | about 0.01%, |
| dimethicone | about 0.42%, |
| methyl GLUCETH-20 | about 42%, |
| hydroxypropylcellulose | about 0.30%, |
| hydroxypropyl methylcellulose | about 0.30%, |
| petrolatum | about 1%, |
| titanium dioxide | about 0.20% and |
| optional phospholipids | about 0.01% (if present). |

In some of the embodiments of the method of the present invention, the massaging in step (b) is preferably performed with damp fingers to promote heat generation. Preferably, the massaging in step (b) is performed for about 20 seconds to about 3 minutes, more preferably about 0.5 minute to about 2 minutes, and even more preferably about 1 minute, to activate heat.

In some of the embodiments of the method of the present invention, the self-heating mask composition is set on the area of the skin in step (c) for preferably about 3 minutes to about 30 minutes, more preferably about 5 minutes to about 20 minutes, and even more preferably about 10 minutes to about 15 minutes. Without being bound by any theory, it is believed that the setting of the self-heating mask would provide heat and moisture to the area of the skin.

In some of embodiments of the method of the present invention, the optional massaging in step (d) is performed for about 20 seconds to about 3 minutes, preferably about 0.5 minute to about 2 minutes, and more preferably about 1 minute.

In some of embodiments of the method of the present invention, the mask is removed from the area of the skin in step (e) preferably with a warm towel, wherein the warm towel may optionally be wet.

In some of embodiments of the method of the present invention, the optional additional skin care product applied in step (f) can be a skin peeling product, e.g., the skin peeling product disclosed in U.S. patent application Ser. No. 09/338,729, filed Jun. 23, 1999, the disclosure of which is incorporated by reference herein.

What is claimed is:

1. A method for administering hyaluronic acid and/or hyaluronate to the skin of a human subject or treating the skin of a human subject comprising
(a) applying an HA liquid to an area of the skin of the human subject, wherein the HA liquid comprises HA, and wherein HA is at least one substance selected from the group consisting of hyaluronic acid, hyaluronate, cosmetically acceptable salts of hyaluronic acid, intramolecular esters of hyaluronic acid and intermolecular esters of hyaluronic acid; and
(b) applying a self-heating mask composition into the area of the skin to generate heat locally, wherein the self-heating mask composition comprises at least one silicoaluminate.

2. The method of claim 1, wherein the self-heating mask composition in step (b) is massaged into the area of the skin to promote heat generation.

3. The method of claim 2, further comprising after step (b),
(c) letting the self-heating mask composition set on the area of the skin to form a mask.

4. The method of claim 3, further comprising after step (c),
(d) massaging the mask into the area of the skin.

5. The method of claim 4, further comprising after step (d),
(e) removing the mask from the area of the skin.

6. The method of claim 5, further comprising after step (e),
(f) applying another skin care product to the area of the skin.

7. The method of claim 1, wherein the silicoaluminate is sodium silicoaluminate, potassium silicoaluminate, lithium silicoaluminate, calcium silicoaluminate, magnesium silicoaluminate or zinc silicoaluminate.

8. The method of claim 7, wherein the silicoaluminate is sodium silicoaluminate.

9. The method of claim 1, wherein the HA comprises hyaluronic acid and/or hyaluronate.

10. The method of claim 9, wherein the HA comprises hyaluronate.

11. The method of claim 10, wherein the HA comprises sodium hyaluronate.

12. The method of claim 1, wherein the HA liquid further comprises at least one cosmetically acceptable excipient, and/or at least one cosmetically acceptable vehicle or carrier.

13. The method of claim 12, wherein the at least one cosmetically acceptable vehicle or carrier is water or a cosmetically acceptable aqueous buffer having a pH of about 7.0 to about 7.4.

14. The method of claim 13, wherein the water or aqueous buffer is purified and/or sterile.

15. The method of claim 12, wherein the at least one cosmetically acceptable excipient is selected from the group consisting of surfactant/emulsifying agents, absorbents, antifoaming agents, binders, biological additives, chelating agents, denaturants, preservatives, solubilizing agents, solvents and thickening agents.

16. The method of claim 15, wherein the at least one cosmetically acceptable excipient comprises at least one surfactant/emulsifying agent.

17. The method of claim 1, wherein the HA liquid further comprises at least one additional active ingredient.

18. The method of claim 17, wherein the at least one additional active ingredient is selected from the group consisting of antioxidants, free-radical scavengers, antimicrobial agents, topical analgesics, steroidal anti-inflammatory drugs, anti-acne agents, reducing agents, vitamins, skin protecting agents, skin bleaching agents, skin conditioning agents, skin soothing agents, skin healing agents, green tea extract, P. emblica (Amla), arnica, chamomile extract and cucumber extract.

19. The method of claim 17, wherein the at least one additional active ingredient comprises at least one antioxidant.

20. The method of claim 1, wherein the self-heating mask composition further comprises at least one cosmetically acceptable excipient, and/or at least one cosmetically acceptable vehicle or carrier.

21. The method of claim 20, wherein the at least one cosmetically acceptable vehicle or carrier is water or a cosmetically acceptable aqueous buffer having a pH of about 7.0 to about 7.4.

22. The method of claim 21, wherein the water or aqueous buffer is purified and/or sterile.

23. The method of claim 20, wherein the at least one cosmetically acceptable excipient is selected from the group consisting of surfactant/emulsifying agents, absorbents, antifoaming agents, binders, biological additives, chelating agents, denaturants, preservatives, solubilizing agents, solvents and thickening agents.

24. The method of claim 20, wherein the at least one cosmetically acceptable excipient comprises at least one surfactant/emulsifying agent.

25. The method of claim 1, wherein the self-heating mask composition further comprises at least one additional active ingredient.

26. The method of claim 25, wherein the at least one additional active ingredient is selected from the group consisting of antioxidants, free-radical scavengers, antimicrobial agents, topical analgesics, steroidal anti-inflammatory drugs, anti-acne agents, reducing agents, vitamins, skin protecting agents, skin bleaching agents, skin conditioning agents, skin soothing agents, skin healing agents, green tea extract, P. emblica (Amla), arnica, chamomile extract and cucumber extract.

27. The method of claim 25, wherein the at least one additional active ingredient comprises at least one antioxidant.

28. The method of claim 1, wherein the HA liquid comprises cyclomethicone, dimethiconol, sodium hyaluronate, Camellia sinensis leaf extract, ascorbyl palmitate, ascorbyl glucosamine, tocopheryl acetate, retinyl palmitate, superoxide dismutase, ubiquinone, phospholipids and purified water with the optional inclusion of ascorbic acid.

29. The method of claim 1, wherein the HA liquid comprises the following ingredients:

| Ingredient | Amount (Weight %) |
|---|---|
| cyclomethicone | about 0% to about 99%, |
| dimethiconol | about 0% to about 30%, |
| sodium hyaluronate | about 0.005% to about 99.5%, |
| Camellia sinensis leaf extract | about 0% to about 20%, |
| optional ascorbic acid | about 0% to about 20%, |
| ascorbyl palmitate | about 0% to about 20%, |
| ascorbyl glucosamine | about 0% to about 20%, |
| tocopheryl acetate | about 0% to about 20%, |
| retinyl palmitate | about 0% to about 20%, |
| superoxide dismutase | about 0% to about 1%, |
| ubiquinone | about 0% to about 20%, |
| phospholipids | about 0% to about 20% and |
| purified water | about 0% to about 50%. |

30. The method of claim 29, wherein the HA liquid comprises the following ingredients:

| Ingredient | Amount (Weight %) |
|---|---|
| cyclomethicone | about 5% to about 95%, |
| dimethiconol | about 5% to about 25%, |
| sodium hyaluronate | about 0.01% to about 85%, |
| Camellia sinensis leaf extract | about 0.01% to about 10%, |
| optional ascorbic acid | about 0.01% to about 10% (if present), |
| ascorbyl palmitate | about 0.01% to about 10%, |
| ascorbyl glucosamine | about 0.01% to about 10%, |
| tocopheryl acetate | about 0.01% to about 10%, |
| retinyl palmitate | about 0.01% to about 10%, |
| superoxide dismutase | about 0.0001% to about 0.1%, |
| ubiquinone | about 0.01% to about 10%, |
| phospholipids | about 0.01% to about 10% and |
| purified water | about 0.01% to about 20%. |

31. The method of claim 30, wherein the HA liquid comprises the following ingredients:

| Ingredient | Amount (Weight %) |
|---|---|
| cyclomethicone | about 10% to about 90%, |
| dimethiconol | about 10% to about 20%, |
| sodium hyaluronate | about 0.01% to about 70%, |
| Camellia sinensis | about 0.01% to about 5%, |
| leaf extract | |
| optional ascorbic acid | about 0.01% to about 5% (if present), |
| ascorbyl palmitate | about 0.01% to about 5%, |
| ascorbyl glucosamine | about 0.01% to about 5%, |
| tocopheryl acetate | about 0.01% to about 5%, |
| retinyl palmitate | about 0.01% to about 5%, |
| superoxide dismutase | about 0.0001% to about 0.01%, |
| ubiquinone | about 0.01% to about 5%, |
| phospholipids | about 0.01% to about 5% and |
| purified water | about 0.01% to about 10%. |

32. The method of claim 31, wherein the HA liquid comprises the following ingredients:

| Ingredient | Amount (Weight %) |
|---|---|
| cyclomethicone | about 86.90%, |
| dimethiconol | about 13.00%, |
| sodium hyaluronate | about 0.01%, |
| Camellia sinensis leaf extract | about 0.01%, |
| optional ascorbic acid | about 0.01% (if present), |
| ascorbyl palmitate | about 0.01%, |
| ascorbyl glucosamine | about 0.01%, |
| tocopheryl acetate | about 0.01%, |
| retinyl palmitate | about 0.01%, |
| superoxide dismutase | about 0.0001%, |
| ubiquinone | about 0.02%, |
| phospholipids | about 0.01% and |
| purified water | about 0.01%. |

33. The method of claim 1, wherein the self-heating mask composition comprises sodium silicoaluminate, butylene glycol, PEG-8, Camellia sinensis leaf extract, white tea extract or juice, ascorbyl palmitate, retinyl palmitate, tocopheryl acetate, dimethicone, methyl GLUCETH-20, hydroxypropylcellulose, hydroxypropyl methylcellulose, petrolatum and titanium dioxide, with optional inclusion of phospholipids.

34. The method of claim 1, wherein the self-heating mask composition comprises the following ingredients:

| Ingredient | Amount (weight %) |
|---|---|
| sodium silicoaluminate | about 2% to about 99%, |
| butylene glycol | about 0% to about 98%, |
| PEG-8 | about 0% to about 25%, |
| Camellia sinensis leaf extract | about 0% to about 20%, |
| white tea extract or juice | about 0% to about 20%, |
| ascorbyl palmitate | about 0% to about 20%, |
| retinyl palmitate | about 0% to about 20%, |
| tocopheryl acetate | about 0% to about 20%, |
| dimethicone | about 0% to about 20%, |
| methyl GLUCETH-20 | about 0% to about 20%, |
| hydroxypropylcellulose | about 0% to about 20%, |
| hydroxypropyl methylcellulose | about 0% to about 20%, |
| petrolatum | about 0% to about 20%, |
| titanium dioxide | about 0% to about 20% and |
| optional phospholipids | about 0% to about 20%. |

35. The method of claim 34, wherein the self-heating mask composition comprises the following ingredients:

| Ingredient | Preferred Amount (Weight %) |
|---|---|
| sodium silicoaluminate | about 10% to about 80%, |
| butylene glycol | about 10% to about 80%, |
| PEG-8 | about 0.1% to about 10%, |
| Camellia sinensis leaf extract | about 0.01% to about 10%, |
| white tea extract or juice | about 0.01% to about 10%, |
| ascorbyl palmitate | about 0.01% to about 10%, |
| retinyl palmitate | about 0.01% to about 10%, |
| tocopheryl acetate | about 0.01% to about 10%, |
| dimethicone | about 0.01% to about 10%, |
| methyl GLUCETH-20 | about 0.01% to about 10%, |
| hydroxypropylcellulose | about 0.01% to about 10%, |
| hydroxypropyl methylcellulose | about 0.01% to about 10%, |
| petrolatum | about 0.1% to about 10%, |
| titanium dioxide | about 0.01% to about 10% and |
| optional phospholipids | about 0.01% to about 10% |
| | (if present). |

36. The method of claim 35, wherein the self-heating mask composition comprises the following ingredients:

| Ingredient | More Preferred Amount (Weight %) |
|---|---|
| sodium silicoaluminate | about 20% to about 70%, |
| butylene glycol | about 20% to about 70%, |
| PEG-8 | about 0.5% to about 5%, |
| Camellia sinensis leaf extract | about 0.01% to about 2%, |
| white tea extract or juice | about 0.01% to about 2%, |
| ascorbyl palmitate | about 0.01% to about 2%, |
| retinyl palmitate | about 0.01% to about 2%, |
| tocopheryl acetate | about 0.01% to about 2%, |
| dimethicone | about 0.01% to about 2%, |
| methyl GLUCETH-20 | about 0.01% to about 2%, |
| hydroxypropylcellulose | about 0.01% to about 2%, |
| hydroxypropyl methylcellulose | about 0.01% to about 2%, |
| petrolatum | about 0.5% to about 5%, |
| titanium dioxide | about 0.01% to about 2% and |
| optional phospholipids | about 0.01% to about 2% |
| | (if present). |

37. The method of claim 36, wherein the self-heating mask composition comprises the following ingredients:

| Ingredient | Amount (Weight %) |
|---|---|
| sodium silicoaluminate | about 31.84%, |
| butylene glycol | about 62.02%, |
| PEG-8 | about 3.35%, |
| Camellia sinensis leaf extract | about 0.10%, |
| white tea extract or juice | about 0.01%, |
| ascorbyl palmitate | about 0.01%, |
| retinyl palmitate | about 0.01%, |
| tocopheryl acetate | about 0.01%, |
| dimethicone | about 0.42%, |
| methyl GLUCETH-20 | about 42%, |
| hydroxypropylcellulose | about 0.30%, |
| hydroxypropyl methylcellulose | about 0.30%, |
| petrolatum | about 1%, |
| titanium dioxide | about 0.20% and |
| optional phospholipids | about 0.01% (if present). |

38. The method of claim 2, wherein the self-heating mask composition in step (b) is massaged into the area of the skin with at least one damp finger.

39. The method of claim 2, wherein the self-heating mask composition in step (b) is massaged into the area of the skin for about 20 seconds to about 3 minutes.

40. The method of claim 3, wherein the self-heating mask composition is set on the area of the skin in step (c) for about 3 minutes to about 30 minutes.

41. The method of claim 4, wherein the massaging in step (d) is performed for about 20 seconds to about 3 minutes.
